(19) United States
(12) Patent Application Publication
(10) Pub. No.: US 2023/0337958 A1
(43) Pub. Date: Oct. 26, 2023

(54) RETRACTABLE ELECTROCARDIOGRAM DEVICE

(71) Applicant: Linda King, Anaheim, CA (US)

(72) Inventor: Linda King, Anaheim, CA (US)

(21) Appl. No.: 17/724,801

(22) Filed: Apr. 20, 2022

(51) Int. Cl. A61B 5/339; A61B 5/271; A61B 5/00; A61B 5/30; A61B 5/282

(52) U.S. Cl. CPC A61B 5/303; A61B 5/271; A61B 5/282; A61B 5/339; A61B 5/6823; A61B 5/6824; A61B 5/6828; A61B 2560/0468

(57) ABSTRACT

A retractable electrocardiogram device, including a main body, a plurality of chest electrodes movably disposed within at least a portion of the main body to removably connect to at least a portion of a chest of a user and move from retracted within the main body to at least partially extracted out of the main body in a first position, and move from extracted out of the main body to retracted within the main body in a second position, a plurality of arm electrodes movably disposed within at least a portion of the main body to removably connect to at least a portion of arms of the user and move from retracted within the main body to at least partially extracted out of the main body in the first position, and move from extracted out of the main body to retracted within the main body in the second position, and a plurality of leg electrodes movably disposed within at least a portion of the main body to removably connect to at least a portion of legs of the user and move from retracted within the main body to at least partially extracted out of the main body in the first position, and move from extracted out of the main body to retracted within the main body in the second position.

### BACKGROUND

### 1. Field

The present general inventive concept relates generally to an electrocardiogram, and particularly, to a retractable electrocardiogram device.

### 2. Description of the Related Art

An electrocardiogram (ECG) is a non-invasive medical procedure used to detect electrical signals in a heart of a person and/or an animal. Typically, the ECG is used to diagnose different heart problems, such as abnormal heart rhythms (i.e., arrhythmias) and/or blocked and/or narrow arteries.

Currently, ECG machines have long wires that become easily entangled during use. Moreover, medical staff using the ECG machines leave the ECG machines in rooms and/or hallways at a medical clinic and/or a hospital that increases clutter due to the long wires either draped on the floor and/or the ECG machine. Regardless of location, the entangled wires are problematic for the medical staff especially during an emergency when a patient needs immediate medical attention.

Therefore, there is a need for a retractable electrocardiogram device that reduces necessary storage space for ECG wiring and facilitates setup during use.

### SUMMARY

The present general inventive concept provides a retractable electrocardiogram device.

Additional features and utilities of the present general inventive concept will be set forth in part in the description which follows and, in part, will be obvious from the description, or may be learned by practice of the general inventive concept.

The foregoing and/or other features and utilities of the present general inventive concept may be achieved by providing a retractable electrocardiogram device, including a main body, a plurality of chest electrodes movably disposed within at least a portion of the main body to removably connect to at least a portion of a chest of a user and move from retracted within the main body to at least partially extracted out of the main body in a first position, and move from extracted out of the main body to retracted within the main body in a second position, a plurality of arm electrodes movably disposed within at least a portion of the main body to removably connect to at least a portion of arms of the user and move from retracted within the main body to at least partially extracted out of the main body in the first position, and move from extracted out of the main body to retracted within the main body in the second position, and a plurality of leg electrodes movably disposed within at least a portion of the main body to removably connect to at least a portion of legs of the user and move from retracted within the main body to at least partially extracted out of the main body in the first position, and move from extracted out of the main body to retracted within the main body in the second position.

The retractable electrocardiogram device may further include a retracting button disposed on at least a portion of the main body to move the plurality of chest electrodes, the plurality of arm electrodes, and the plurality of leg electrodes from extracted out of the main body to retracted within the main body in response to being depressed.

The plurality of chest electrodes, the plurality of arm electrodes, and the plurality of leg electrodes may retract in response to being tugged away from the main body while at least partially extended away from the main body.

The retractable electrocardiogram device may further include a display unit disposed on at least a portion of the main body to display a current position of each of the plurality of chest electrodes, each of the plurality of arm electrodes, and each of the plurality of leg electrodes as disposed on a body of the user.

The retractable electrocardiogram device may further include a processing unit disposed within at least a portion of the main body to determine the placement of at least one of the plurality of chest electrodes, the plurality of arm electrodes, and the plurality of leg electrodes in response to receiving placement data from a sensor of each of the plurality of chest electrodes, each of the plurality of arm electrodes, and each of the plurality of leg electrodes.

### BRIEF DESCRIPTION OF THE DRAWINGS

These and/or other features and utilities of the present generally inventive concept will become apparent and more readily appreciated from the following description of the embodiments, taken in conjunction with the accompanying drawings of which:

FIG. 1 illustrates a front perspective view of a retractable electrocardiogram device, according to an exemplary embodiment of the present general inventive concept.

### DETAILED DESCRIPTION

Various example embodiments (a.k.a., exemplary embodiments) will now be described more fully with reference to the accompanying drawings in which some example embodiments are illustrated. In the FIGURE, the thicknesses of lines, layers and/or regions may be exaggerated for clarity.

Accordingly, while example embodiments are capable of various modifications and alternative forms, embodiments thereof are shown by way of example in the FIGURE and will herein be described in detail. It should be understood, however, that there is no intent to limit example embodiments to the particular forms disclosed, but on the contrary, example embodiments are to cover all modifications, equivalents, and alternatives falling within the scope of the disclosure. Like numbers refer to like/similar elements throughout the detailed description.

It is understood that when an element is referred to as being “connected” or “coupled” to another element, it can be directly connected or coupled to the other element or intervening elements may be present. In contrast, when an element is referred to as being “directly connected” or “directly coupled” to another element, there are no intervening elements present. Other words used to describe the relationship between elements should be interpreted in a like fashion (e.g., “between” versus “directly between,” “adjacent” versus “directly adjacent,” etc.).

The terminology used herein is for the purpose of describing particular embodiments only and is not intended to be limiting of example embodiments. As used herein, the singular forms “a,” “an” and “the” are intended to include the plural forms as well, unless the context clearly indicates otherwise. It will be further understood that the terms “comprises,” “comprising,” “includes” and/or “including,” when used herein, specify the presence of stated features, integers, steps, operations, elements and/or components, but do not preclude the presence or addition of one or more other features, integers, steps, operations, elements, components and/or groups thereof.

Unless otherwise defined, all terms (including technical and scientific terms) used herein have the same meaning as commonly understood by one of ordinary skill in the art to which example embodiments belong. It will be further understood that terms, e.g., those defined in commonly used dictionaries, should be interpreted as having a meaning that is consistent with their meaning in the context of the relevant art. However, should the present disclosure give a specific meaning to a term deviating from a meaning commonly understood by one of ordinary skill, this meaning is to be taken into account in the specific context this definition is given herein.

### List of Components

Retractable Electrocardiogram Device 100
Main Body 110
Chest Electrodes 120
V1 electrode 121
V2 electrode 122
V3 electrode 123
V4 electrode 124
V5 electrode 125
V6 electrode 126
Arm Electrodes 130
Right Arm Electrode 131
Left Arm Electrode 132
Leg Electrodes 140
Right Leg Electrode 141
Left Leg Electrode 142
Retracting Button 150
Display Unit 160
Processing Unit 170
ECG Connector 180

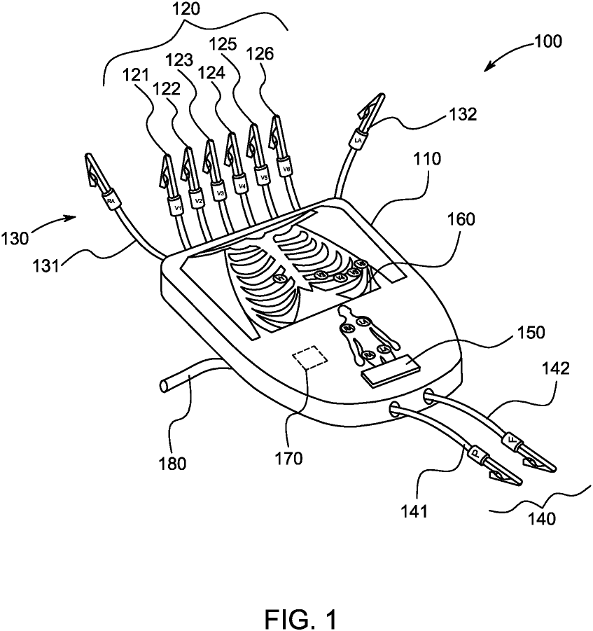

FIG. 1 illustrates a front perspective view of a retractable electrocardiogram device 100, according to an exemplary embodiment of the present general inventive concept.

The retractable electrocardiogram device 100 may be constructed from at least one of metal, plastic, wood, glass, and rubber, etc., but is not limited thereto.

The retractable electrocardiogram device 100 may include a main body 110, a plurality of chest electrodes 120, a plurality of arm electrodes 130, a plurality of leg electrodes 140, a retracting button 150, a display unit 160, a processing unit 170, and an ECG connector 180, but is not limited thereto.

The main body 110 may be disposed on a body of a user (e.g., a patient) and/or a surface (e.g., a table, a desk). Alternatively, the main body 110 may be suspended on a rack, such as a medical cart.

The plurality of chest electrodes 120 may include a V1 electrode 121, a V2 electrode 122, a V3 electrode 123, a V4 electrode 124, a V5 electrode 125, and a V6 electrode 126, but is not limited thereto.

The V1 electrode 121, the V2 electrode 122, the V3 electrode 123, the V4 electrode 124, the V5 electrode 125, and/or the V6 electrode 126 may be movably (i.e., springingly) disposed within at least a portion of the main body 110. More specifically, the V1 electrode 121, the V2 electrode 122, the V3 electrode 123, the V4 electrode 124, the V5 electrode 125, and/or the V6 electrode 126 may move from retracted within the main body 110 to at least partially extracted out of the main body 110 in a first position. Conversely, the V1 electrode 121, the V2 electrode 122, the V3 electrode 123, the V4 electrode 124, the V5 electrode 125, and/or the V6 electrode 126 may move from extracted out of the main body 110 to retracted within the main body 110 in a second position.

Also, the V1 electrode 121, the V2 electrode 122, the V3 electrode 123, the V4 electrode 124, the V5 electrode 125, and/or the V6 electrode 126 may be spring biased (e.g., via a spring) to retract within the main body 110.

The V1 electrode 121, the V2 electrode 122, the V3 electrode 123, the V4 electrode 124, the V5 electrode 125, and/or the V6 electrode 126 may be removably connected to least a portion of a chest of the user. For example, the V1 electrode 121 may be connected to a fourth intercostal space to the right of a sternum, the V2 electrode 122 may be connected to the fourth intercostal space to the left of the sternum, the V3 electrode 123 may be connected to the chest between the V2 electrode 122 and V4 electrode 124, the V4 electrode 124 may be connected to a fifth intercostal space at a nipple line, the V5 electrode 125 may be connected between the V4 electrode 124 and the V6 electrode 126, and/or the V6 electrode 126 may be connected to a midaxillary line on same height as the V4 electrode 124,

The plurality of arm electrodes 130 may include a right arm electrode 131 and a left arm electrode 132, but is not limited thereto.

The right arm electrode 131 and/or the left arm electrode 132 may be movably (i.e., springingly) disposed within at least a portion of the main body 110. More specifically, the right arm electrode 131 and/or the left arm electrode 132 may move from retracted within the main body 110 to at least partially extracted out of the main body 110 in a first position. Conversely, the right arm electrode 131 and/or the left arm electrode 132 may move from extracted out of the main body 110 to retracted within the main body 110 in a second position.

Also, the right arm electrode 131 and/or the left arm electrode 132 may be spring biased (e.g., via a spring) to retract within the main body 110.

The right arm electrode 131 and/or the left arm electrode 132 may be removably connected to least a portion of a right arm and/or a left arm, respectively, of the user.

The plurality of leg electrodes 140 may include a right leg electrode 141 and a left leg electrode 142, but is not limited thereto.

The right leg electrode 141 and/or the left leg electrode 142 may be movably (i.e., springingly) disposed within at least a portion of the main body 110. More specifically, the right leg electrode 141 and/or the left leg electrode 142 may move from retracted within the main body 110 to at least partially extracted out of the main body 110 in a first position. Conversely, the right leg electrode 141 and/or the left leg electrode 142 may move from extracted out of the main body 110 to retracted within the main body 110 in a second position.

Also, the right leg electrode 141 and/or the left leg electrode 142 may be spring biased (e.g., via a spring) to retract within the main body 110.

The right leg electrode 141 and/or the left leg electrode 142 may be removably connected to least a portion of a right leg and/or a left leg, respectively, of the user.

Furthermore, each of the plurality of chest electrodes 120, each of the plurality of arm electrodes 130, and/or each of the plurality of leg electrodes 140 may each have a sensor that identifies a current position on the body of the user.

It is important to note that the plurality of chest electrodes 120, the plurality of arm electrodes 130, the plurality of leg electrodes 140, and/or the display unit 160 may be extracted in response to an external application of force (e.g., pushing, pulling).

The retracting button 150 may be disposed on at least a portion of the main body 110 and/or connected to the plurality of chest electrodes 120, the plurality of arm electrodes 130, and/or the plurality of leg electrodes 140. Additionally, the plurality of chest electrodes 120, the plurality of arm electrodes 130, and/or the plurality of leg electrodes 140 may move from extracted out of the main body 110 to retracted within the main body 110 in response to depressing the retracting button 150. In other words, the retracting button 150 may trigger the spring to retract the plurality of chest electrodes 120, the plurality of arm electrodes 130, and/or the plurality of leg electrodes 140 within the main body 110. Alternatively, the plurality of chest electrodes 120, the plurality of arm electrodes 130, and/or the plurality of leg electrodes 140 may retract in response to tugging each of the plurality of chest electrodes 120, each of the plurality of arm electrodes 130, and/or each of the plurality of leg electrodes 140 away from the main body 110 to trigger the spring while at least partially extended away from the main body 110.

The display unit 160 may include a plasma screen, an LCD screen, a light emitting diode (LED) screen, an organic LED (OLED) screen, a computer monitor, a hologram output unit, a sound outputting unit, or any other type of device that visually or aurally displays data. Also, the display unit 160 may be a touch-screen that receives an input thereon. Alternatively, the display unit 160 may be indicia to identify a location of placement of the plurality of chest electrodes 120, the plurality of arm electrodes 130, and/or the plurality of leg electrodes 140 on a body of the user.

The display unit 160 may be disposed on at least a portion of the main body 110. The display unit 160 may display the current position of each of the plurality of chest electrodes 120, each of the plurality of arm electrodes 130, and/or each of the plurality of leg electrodes 140. As such, the display unit 160 may identify incorrect placement based on a comparison to a proper position identifier, such that the plurality of chest electrodes 120, the plurality of arm electrodes 130, and/or the plurality of leg electrodes 140 may be moved to proper location on the body of the user.

The processing unit 170 (or central processing unit, CPU) may include electronic circuitry to carry out instructions of a computer program by performing basic arithmetic, logical, control and input/output (I/O) operations specified by the instructions. The processing unit 170 may include an arithmetic logic unit (ALU) that performs arithmetic and logic operations, processor registers that supply operands to the ALU and store the results of ALU operations, and a control unit that fetches instructions from memory and “executes” them by directing the coordinated operations of the ALU, registers and other components. The processing unit 170 may also include a microprocessor and a microcontroller.

The processing unit 170 may be disposed within at least a portion of the main body 110 and/or connected to the plurality of chest electrodes 120, the plurality of arm electrodes 130, the plurality of leg electrodes 140, and/or the display unit 160. The processing unit 170 may determine the placement of the plurality of chest electrodes 120, the plurality of arm electrodes 130, and/or the plurality of leg electrodes 140 in response to receiving placement data from each sensor. As such, the processing unit 170 may send the current position of each of the plurality of chest electrodes 120, each of the plurality of arm electrodes 130, and/or each of the plurality of leg electrodes 140 to the display unit 160.

The ECG connector 180 may be disposed on at least a portion of the main body 110. The ECG connector 180 may connect to an external device (e.g., a computer, an ECG machine, a mobile device) to transmit data received by the plurality of chest electrodes 120, the plurality of arm electrodes 130, and/or the plurality of leg electrodes 140 to the external device.

Therefore, the retractable electrocardiogram device 100 may reduce clutter during storage and/or facilitate setup during use. Also, the retractable electrocardiogram device 100 may save lives due to less confusion required to disentangle the electrodes prior to use.

The present general inventive concept may include a retractable electrocardiogram device 100, including a main body 110, a plurality of chest electrodes 120 movably disposed within at least a portion of the main body 110 to removably connect to at least a portion of a chest of a user and move from retracted within the main body 110 to at least partially extracted out of the main body 110 in a first position, and move from extracted out of the main body 110 to retracted within the main body 110 in a second position, a plurality of arm electrodes 130 movably disposed within at least a portion of the main body 110 to removably connect to at least a portion of arms of the user and move from retracted within the main body 110 to at least partially extracted out of the main body 110 in the first position, and move from extracted out of the main body 110 to retracted within the main body 110 in the second position, and a plurality of leg electrodes 140 movably disposed within at least a portion of the main body 110 to removably connect to at least a portion of legs of the user and move from retracted within the main body 110 to at least partially extracted out of the main body 110 in the first position, and move from extracted out of the main body 110 to retracted within the main body 110 in the second position.

The retractable electrocardiogram device 100 may further include a retracting button 150 disposed on at least a portion of the main body 110 to move the plurality of chest electrodes 120, the plurality of arm electrodes 130, and the plurality of leg electrodes 140 from extracted out of the main body 110 to retracted within the main body 110 in response to being depressed.

The plurality of chest electrodes 120, the plurality of arm electrodes 130, and the plurality of leg electrodes 140 may retract in response to being tugged away from the main body 110 while at least partially extended away from the main body 110.

The retractable electrocardiogram device 100 may further include a display unit 160 disposed on at least a portion of the main body 110 to display a current position of each of the plurality of chest electrodes 120, each of the plurality of arm electrodes 130, and each of the plurality of leg electrodes 140 as disposed on a body of the user.

The retractable electrocardiogram device 100 may further include a processing unit 170 disposed within at least a portion of the main body 110 to determine the placement of at least one of the plurality of chest electrodes 120, the plurality of arm electrodes 130, and the plurality of leg electrodes 140 in response to receiving placement data from a sensor of each of the plurality of chest electrodes 120, each of the plurality of arm electrodes 130, and each of the plurality of leg electrodes 140.

Although a few embodiments of the present general inventive concept have been shown and described, it will be appreciated by those skilled in the art that changes may be made in these embodiments without departing from the principles and spirit of the general inventive concept, the scope of which is defined in the appended claims and their equivalents.

1. A retractable electrocardiogram device, comprising:
a main body;
a plurality of chest electrodes movably disposed within at least a portion of the main body to removably connect to at least a portion of a chest of a user and move from retracted within the main body to at least partially extracted out of the main body in a first position, and move from extracted out of the main body to retracted within the main body in a second position;
a plurality of arm electrodes movably disposed within at least a portion of the main body to removably connect to at least a portion of arms of the user and move from retracted within the main body to at least partially extracted out of the main body in the first position, and move from extracted out of the main body to retracted within the main body in the second position; and
a plurality of leg electrodes movably disposed within at least a portion of the main body to removably connect to at least a portion of legs of the user and move from retracted within the main body to at least partially extracted out of the main body in the first position, and move from extracted out of the main body to retracted within the main body in the second position.

2. The retractable electrocardiogram device of claim 1, further comprising:
a retracting button disposed on at least a portion of the main body to move the plurality of chest electrodes, the plurality of arm electrodes, and the plurality of leg electrodes from extracted out of the main body to retracted within the main body in response to being depressed.

3. The retractable electrocardiogram device of claim 1, wherein the plurality of chest electrodes, the plurality of arm electrodes, and the plurality of leg electrodes retract in response to being tugged away from the main body while at least partially extended away from the main body.

4. The retractable electrocardiogram device of claim 1, further comprising:
a display unit disposed on at least a portion of the main body to display a current position of each of the plurality of chest electrodes, each of the plurality of arm electrodes, and each of the plurality of leg electrodes as disposed on a body of the user.

5. The retractable electrocardiogram device of claim 1, further comprising:
a processing unit disposed within at least a portion of the main body to determine the placement of at least one of the plurality of chest electrodes, the plurality of arm electrodes, and the plurality of leg electrodes in response to receiving placement data from a sensor of each of the plurality of chest electrodes, each of the plurality of arm electrodes, and each of the plurality of leg electrodes.
